SEQUENCE LISTING (8 sequences)

SEQ ID NO: 1 - RNA, 23 nt, Homo sapiens
ugguuguaguccgugcgagaaua

SEQ ID NO: 2 - RNA, 70 nt, Homo sapiens
uguucuuguaguugaaauacaacgaugguuuuucauaucauuggucgugguuguaguccgugcgagaaua

SEQ ID NO: 3 - RNA, 22 nt, Homo sapiens
gauuagggugcuuagcuguuaa

SEQ ID NO: 4 - RNA, 79 nt, Homo sapiens
uugauuagggugcuuagcuguuaacuaaguguuuguggguuuaagucccauuggucuaguaagggcuuagcuuaauuaa

SEQ ID NO: 5 - RNA, 21 nt, Homo sapiens
gguuugguccuagccuuucua

SEQ ID NO: 6 - RNA, 53 nt, Homo sapiens
uagacgggcucacaucaccccauaaacaaauagguuugguccuagccuuucua

SEQ ID NO: 7 - RNA, 24 nt, Homo sapiens
agccugauuaaacacaugcucuga

SEQ ID NO: 8 - RNA, 85 nt, Homo sapiens
uuuacaguuugccaugaugaaaugcauguuaaguccguguuucagcugaucagccugauuaaacacaugcucugagcagacuaaa

(19) United States
(12) Patent Application Publication
(10) Pub. No.: US 2014/0134728 A1
(43) Pub. Date: May 15, 2014

(54) METHODS FOR ADJUSTING EXPRESSION OF MITOCHONDRIAL GENOME BY MICRORNA

(71) Applicants: Alexandra Henrion-Caude, Paris (FR); Simonetta Bandiera, Paris (FR); Stanislas Lyonnet, Paris (FR)

(72) Inventors: Alexandra Henrion-Caude, Paris (FR); Simonetta Bandiera, Paris (FR); Stanislas Lyonnet, Paris (FR)

(73) Assignee: INSERM (Institut National de la Sante et de la Recherche Medicale), Paris (FR)

(21) Appl. No.: 14/122,716

(22) PCT Filed: Jun. 1, 2012
(86) PCT No.: PCT/EP2012/060336

§ 371 Date: Jan. 9, 2014

Related U.S. Application Data
(60) Provisional application No. 61/492,058, filed on Jun. 1, 2011.

(51) Int. Cl. C12N 15/113

(52) U.S. Cl. CPC C12N 15/113
USPC 435/366

(57) ABSTRACT

The present invention relates to a method for adjusting the expression of the mitochondrial genome in a human cell comprising a step consisting of modulating the expression of a least one mi RNA selected from the group consisting of hsa-mi R-1973, hsa-mi R-1275, hsa-mi R-494, hsa-mi R-513a-5p, hsa-mi R-1246, hsa-mi R-328, hsa-mi R-1908, hsa-mi R-1972, hsa-mi R-1974, hsa-mi R-1977, hsa-mi R-638, hsa-mi R-1978 and hsa-mi R-1201.

### FIELD OF THE INVENTION

The present invention relates to methods of adjusting expression of mirochondrial genome.

### BACKGROUND OF THE INVENTION

Mitochondria are eukaryotic organelles that maintain and express their own genome, known as the mitochondrial DNA (mtDNA). The transcription and translation of the mtDNA as well as the processing of mitochondrial transcripts requires the involvement of several types of non-coding RNAs (ncRNA), which can be either mitochondrially encoded or transcribed within the nucleus and subsequently localized to mitochondria [1]. In human mitochondria, the full set of mitochondrial transfer RNAs (tRNAs) and two ribosomal RNAs (rRNAs), namely the 12S and 16S rRNAs, are transcribed from the mtDNA [2], while the RNA moiety of the RNase MRP enzymes [3,4,5], the 5S rRNA [6,7], and two species of tRNAGln [8] are all RNAs delivered into mitochondria from the nucleus.

Among ncRNAs, microRNAs (miRNAs) have emerged as an important class of post-transcriptional regulators of gene expression in virtually all fundamental cellular processes [9]. miRNAs are transcribed within the nucleus and are then extensively processed and matured in the cytosol as ˜22-bp double-stranded RNA. Mature miRNAs associate with Argonaute (AGO) proteins to form the core of a ribonucleoprotein complex named RNA-induced silcencing complex (RISC), which exerts RNA interference (RNAi) [10,11]. RNAi occurs upon pairing one of the two miRNA strands, embedded in an AGO protein, with target sites in an mRNA, thereby affecting the stability/translation of this mRNA [11]. In mammals, there are four AGO proteins, AGO1 through AGO4, which were all shown to function in translational repression [12], but only AGO2 can catalyze the cleavage of the targeted transcript [13,14]. Furthermore, knockdown and knockout of AGO2, respectively in human cells and in mice, suggest that the protein may have specific functions that may not be complemented by the other AGOs [13,15].

Initially, mature miRNAs and AGO2 were believed to accumulate and function exclusively in the cytosol and/or into unstructured cytosolic foci, such as P-bodies and stress granules [16,17]. However, mounting evidence suggests that they can also localize to and, possibly, function within different cellular compartments. So far, in human, miRNAs and AGO2 have been found to localize to the nucleus [18,19,20] and to multivesicular bodies [21,22]. Recently, miRNAs were also identified in mitochondria purified from rat liver [23]. Interestingly, another possible link between mitochondria and RNAi came from the co-immunoprecipitation of human AGO2 with mitochondrial tRNAMet [24].

In mitochondria, post-transcriptional regulation via miRNAs would provide a sensitive and rapid mechanism by which to adjust the expression of the mitochondrial genome in relation to the conditions and metabolic demands of the human cell. However the localization of nuclear-encoded miRNAs has not yet been investigated in human cells.

### SUMMARY OF THE INVENTION

The present invention relates to a method for adjusting the expression of the mitochondrial genome in a human cell comprising a step consisting of modulating the expression of a least one miRNA selected from the group consisting of hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201.

### DETAILED DESCRIPTION OF THE INVENTION

MicroRNAs (miRNAs) are small non-coding RNAs that associate with Argonaute proteins to regulate gene expression at the post-transcriptional level in the cytoplasm. However, recent studies have reported that some miRNAs localize to and function in other cellular compartments. Mitochondria harbour their own genetic system that may be a potential site for miRNA mediated post-transcriptional regulation. The inventors aimed at investigating whether nuclear-encoded miRNAs can localize to and function in human mitochondria.

To enable identification of mitochondrial-enriched miRNAs, the inventors profiled the mitochondrial and cytosolic RNA fractions from the same HeLa cells by miRNA microarray analysis. Mitochondria were purified using a combination of human cell fractionation and immunoisolation, and assessed for the lack of protein and RNA contaminants. The inventors found 57 miRNAs differentially expressed in HeLa mitochondria and cytosol. Of these 57, a signature of 13 nuclear-encoded miRNAs was reproducibly enriched in mitochondrial RNA and validated by RT-PCR for hsa-miR-494, hsa-miR-1275 and hsa-miR-1974. The significance of their mitochondrial localization was investigated by characterizing their genomic context, cross-species conservation and instrinsic features such as their size and thermodynamic parameters. Interestingly, the specificities of mitochondrial versus cytosolic miRNAs were underlined by significantly different structural and thermodynamic parameters. Computational targeting analysis of most mitochondrial miRNAs revealed not only nuclear but also mitochondrial-encoded targets. The functional relevance of miRNAs in mitochondria was supported by the finding of Argonaute 2 localization to mitochondria revealed by immunoblotting and confocal microscopy, and further validated by the co-immunoprecipitation of the mitochondrial transcript COX3.

Accordingly the present invention provides the first comprehensive view of the localization of RNA interference components to the mitochondria. The data outline the molecular bases for a novel layer of crosstalk between nucleus and mitochondria through a specific subset of human miRNAs that the inventors termed ‘mitomiRs’.

The present invention relates to a method for adjusting the expression of the mitochondrial genome in a human cell comprising a step consisting of modulating the expression of a least one miRNA selected from the group consisting of hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201.

All the miRNAs pertaining to the invention are known per se and sequences of them are publicly available from the data base http://www.mirbase.org/cgi-bin/mirna_summary.pl?org=hsa excepting for hsa-miR-1974, hsa-miR-1977, hsa-miR-1978 and hsa-miR-1201 which sequences are described in Table A:

TABLE A

| Name | | |
|---|---|---|
| of | Mature | |
| miRNA | sequence | Stem-loop miRNA sequence |
| hsa- | UGGUUGUAG | UGUUCUUGUAGUUGAAAUACAACGAUGGUUUUUCA |
| miR- | UCCGUGCGAG | UAUCAUUGGUCGUGGUUGUAGUCCGUGCGAGAAUA |
| 1974 | AAUA | |
| hsa- | GAUUAGGGU | UUGAUUAGGGUGCUUAGCUGUUAACUAAGUGUUU |
| miR- | GCUUAGCUG | GUGGGUUUAAGUCCCAUUGGUCUAGUAAGGGCUUA |
| 1977 | UUAA | GCUUAAUUAA |
| hsa- | GGUUUGGUC | UAGACGGGCUCACAUCACCCCAUAAACAAAUAGGU |
| miR- | CUAGCCUUUC | UUGGUCCUAGCCUUUCUA |
| 1978 | UA | |
| hsa- | AGCCUGAUU | UUUACAGUUUGCCAUGAUGAAAUGCAUGUUAAGUC |
| miR- | AAACACAUGC | CGUGUUUCAGCUGAUCAGCCUGAUUAAACACAUGC |
| 1201 | UCUGA | UCUGAGCAGACUAAA |

The miRNAs of the invention namely, hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201 are referred to as mitomiR

According to the invention, the method of the present invention may be applied in vitro or in vivo.

The step consisting of modulating the expression of at least one miRNA of the invention may be performed with a compound that inhibit the expression of said miRNA or a compound that raises the expression of said miRNA.

In a particular embodiment, the compound that raises the expression level of one miRNA selected from the group consisting hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201 may consist in an isolated miRNA selected from the group consisting of isolated hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201.

As used herein, an “isolated” miRNA is one which is synthesized, or altered or removed from the natural state through human intervention. For example, a miRNA naturally present in a living animal is not “isolated.” A synthetic miRNA, or a miRNA partially or completely separated from the coexisting materials of its natural state, is “isolated.” An isolated miRNA can exist in substantially purified form, or can exist in a human cell into which the miRNA has been delivered. Thus, a miRNA which is deliberately delivered to, or expressed in, a human cell is considered an “isolated” miRNA. A miRNA produced inside a human cell by from a miRNA precursor molecule is also considered to be “isolated” molecule.

Isolated miRNAs can be obtained using a number of standard techniques. For example, the miRNAs can be chemically synthesized or recombinantly produced using methods known in the art. Preferably, miRNAs are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

In some embodiments, of the invention, a synthetic miRNA contains one or more design elements. These design elements include, but are not limited to: (i) a replacement group for the phosphate or hydroxyl of the nucleotide at the 5′ terminus of the complementary region; (ii) one or more sugar modifications. In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. In particular embodiments, the sugar modification is a 2′O-Me modification. In further embodiments, there is one or more sugar modifications in the first or last 2 to 4 residues of the complementary region or the first or last 4 to 6 residues of the complementary region.

Alternatively, the miRNAs can be expressed from recombinant circular or linear DNA plasmids using any suitable promoter. Suitable promoters for expressing RNA from a plasmid include, e.g., the U6 or HI RNA pol III promoter sequences, or the cytomegalovirus promoters. Selection of other suitable promoters is within the skill in the art. The recombinant plasmids of the invention can also comprise inducible or regulatable promoters for expression of the miRNAs in cells.

The miRNAs that are expressed from recombinant plasmids can be isolated from cultured human cell expression systems by standard techniques. The miRNAs which are expressed from recombinant plasmids can also be delivered to, and expressed directly in, the cells. The use of recombinant plasmids to deliver the miRNAs to cells is discussed in more detail below.

The miRNAs can be expressed from a separate recombinant plasmid, or can be expressed from the same recombinant plasmid. Preferably, the miRNAs are expressed as the RNA precursor molecules from a single plasmid, and the precursor molecules are processed into the functional miRNA by a suitable processing system, including processing systems extant within a human cell. Other suitable processing systems include, e.g., the in vitro Drosophila human cell lysate system as described in U.S. published application 2002/0086356 to Tuschl et al. and the E. coli RNAse III system described in U.S. published patent application 2004/0014113 to Yang et al., the entire disclosures of which are herein incorporated by reference.

Selection of plasmids suitable for expressing the miRNAs, methods for inserting nucleic acid sequences into the plasmid to express the gene products, and methods of delivering the recombinant plasmid to the cells of interest are within the skill in the art. See, for example, Zeng et al. (2002), Molecular Human cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are herein incorporated by reference.

In one embodiment, a plasmid expressing the miRNAs comprises a sequence encoding a miR precursor RNA under the control of the CMV intermediate early promoter. As used herein, “under the control” of a promoter means that the nucleic acid sequences encoding the miRNA are located 3′ of the promoter, so that the promoter can initiate transcription of the miRNA coding sequences.

The miRNAs can also be expressed from recombinant viral vectors. It is contemplated that the miRNAs can be expressed from separate recombinant viral vectors, or from the same viral vector. The RNA expressed from the recombinant viral vectors can either be isolated from cultured human cell expression systems by standard techniques, or can be expressed directly in cells. The use of recombinant viral vectors to deliver the miRNAs to cells is discussed in more detail below.

The recombinant viral vectors of the invention comprise sequences encoding the miRNAs and any suitable promoter for expressing the RNA sequences. Suitable promoters include, for example, the U6 or HI RNA pol III promoter sequences, or the cytomegalovirus promoters. Selection of other suitable promoters is within the skill in the art. The recombinant viral vectors of the invention can also comprise inducible or regulatable promoters for expression of the miRNAs in a human cell.

Any viral vector capable of accepting the coding sequences for the miRNAs can be used; for example, vectors derived from adenovirus (AV); adenoassociated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate. For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J. E. et al. (2002), J Virol 76:791801, the entire disclosure of which is herein incorporated by reference.

Selection of recombinant viral vectors suitable for use in the invention, methods for inserting nucleic acid sequences for expressing RNA into the vector, methods of delivering the viral vector to the cells of interest, and recovery of the expressed RNA products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are herein incorporated by reference.

Preferred viral vectors are those derived from AV and AAV. A suitable AV vector for expressing the miRNAs, a method for constructing the recombinant AV vector, and a method for delivering the vector into target cells, are described in Xia et al. (2002), Nat. Biotech. 20:1006-1010, the entire disclosure of which is herein incorporated by reference. Suitable AAV vectors for expressing the miRNAs, methods for constructing the recombinant AAV vector, and methods for delivering the vectors into target cells are described in Samulski et al. (1987), J. Virol. 61:3096-3101; Fisher et al. (1996), J. Virol., 70:520-532; Samulski et al. (1989), J. Virol. 63:3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference. Preferably, the miRNAs are expressed from a single recombinant AAV vector comprising the CMV intermediate early promoter.

In one embodiment, a recombinant AAV viral vector of the invention comprises a nucleic acid sequence encoding an miR precursor RNA in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the miRNA sequences from the vector, the polyT termination signals act to terminate transcription.

Suitable compounds for inhibiting miRNA expression include double-stranded RNA (such as short- or small-interfering RNA or “siRNA”), antisense nucleic acids, and enzymatic RNA molecules such as ribozymes. Each of these compounds can be targeted to a given miRNA and destroy or induce the destruction of the target miRNA. For example, expression of a given miRNA can be inhibited by inducing RNA interference of the miRNA with an isolated double-stranded RNA (“dsRNA”) molecule which has at least 90%, for example 95%, 98%, 99% or 100%, sequence homology with at least a portion of the miRNA. In a preferred embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target miRNA.

As used herein, a nucleic acid sequence in an siRNA which is “substantially identical” to a target sequence contained within the target mRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area. The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA can also comprise a 3′ overhang. As used herein, a “3″ overhang” refers to at least one unpaired nucleotide extending from the 3′-end of a duplexed RNA strand. Thus, in one embodiment, the siRNA comprises at least one 3′ overhang of from 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length. In a preferred embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated miRNAs. Exemplary methods for producing and testing dsRNA or siRNA molecules are described in U.S. published patent application 2002/0173478 to Gewirtz and in U.S. published patent application 2004/0018176 to Reich et al., the entire disclosures of which are herein incorporated by reference.

Expression of a given miRNA can also be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in an miRNA. Preferably, the antisense nucleic acid comprises a nucleic acid sequence that is 50-100% complementary, more preferably 75-100% complementary, and most preferably 95-100% complementary to a contiguous nucleic acid sequence in an miRNA. Nucleic acid sequences for the miRNAs are provided in Table A. Without wishing to be bound by any theory, it is believed that the antisense nucleic acids activate RNase H or some other cellular nuclease that digests the miRNA/antisense nucleic acid duplex.

Antisense nucleic acids can also contain modifications to the nucleic acid backbone or to the sugar and base moieties (or their equivalent) to enhance target specificity, nuclease resistance, delivery or other properties related to efficacy of the molecule. Such modifications include cholesterol moieties, duplex intercalators such as acridine or the inclusion of one or more nuclease-resistant groups.

Antisense nucleic acids can be produced chemically or biologically, or can be expressed from a recombinant plasmid or viral vector, as described above for the isolated miRNAs. Exemplary methods for producing and testing are within the skill in the art; see, e.g., Stein and Cheng (1993), Science 261:1004 and U.S. Pat. No. 5,849,902 to Woolf et al., the entire disclosures of which are herein incorporated by reference.

Expression of a given miRNA can also be inhibited by an enzymatic nucleic acid. As used herein, an “enzymatic nucleic acid” refers to a nucleic acid comprising a substrate binding region that has complementarity to a contiguous nucleic acid sequence of an miRNA, and which is able to specifically cleave the miRNA. Preferably, the enzymatic nucleic acid substrate binding region is 50-100% complementary, more preferably 75-100% complementary, and most preferably 95-100% complementary to a contiguous nucleic acid sequence in an miRNA. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated miRNAs. Exemplary methods for producing and testing dsRNA or siRNA molecules are described in Werner and Uhlenbeck (1995), Nucl. Acids Res. 23:2092-96; Hammann et al. (1999), Antisense and Nucleic Acid Drug Dev. 9:25-31; and U.S. Pat. No. 4,987,071 to Cech et al, the entire disclosures of which are herein incorporated by reference.

The method of the invention is particularly suitable for adjusting the mitochondrial homeostasis and thus may represent a therapeutic tool for the treatment of various diseases associated with mitochondrial dysfunction. Diseases associated with mitochondrial dysfunction includes any condition or disorder which is directly or indirectly caused by mitochondrial dysfunction and may affect any organ in the human body such as brain, nerves, muscles, heart, eyes, kidneys, lung, liver . . . . These diseases include, by way of example and not limitation, chronic neurodegenerative disorders such as Alzheimer's disease (AD) and Parkinson's disease (PD); auto-immune diseases; diabetes mellitus, including Type I and Type II; mitochondria associated diseases, including but not limited to congenital muscular dystrophy with mitochondrial structural abnormalities, fatal infantile myopathy with severe mtDNA depletion and benign “later-onset” myopathy with moderate reduction in mtDNA, MELAS (mitochondrial encephalopathy, lactic acidosis, and stroke) and MIDD (mitochondrial diabetes and deafness); MERFF (myoclonic epilepsy ragged red fiber syndrome); arthritis; NARP (Neuropathy; Ataxia; Retinitis Pigmentosa); MNGIE (Myopathy and external ophthalmoplegia; Neuropathy; Gastro-Intestinal; Encephalopathy), LHON (leber's; Hereditary; Optic; Neuropathy), Kearns-Sayre disease; Pearson's Syndrome; PEO (Progressive External Ophthalmoplegia); Wolfram syndrome DIDMOAD (Diabetes Insipidus, Diabetes Mellitus, Optic Atrophy, Deafness); Leigh's Syndrome; dystonia; schizophrenia; hyperproliferative disorders, such as cancer, tumors and psoriasis and cardiovascular diseases. Treatment of aging is also encompassed in the scope of the invention.

Accordingly, the present invention also a compound that raises the expression of at least one miRNA selected from the group consisting of hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201 for use in a method for adjusting mitochondrial homeostasis in a subject in need thereof.

A further object of the invention also relates to a compound that a compound that inhibit the expression of at least one miRNA selected from the group consisting of hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201 for use in a method for adjusting mitochondrial homeostasis in a subject in need thereof.

Typically, said subject is affected with a disease associated with a mitochondrial dysfunction.

One skilled in the art can readily determine an effective amount of said compound to be administered to a given subject, by taking into account factors such as the size and weight of the subject; the extent of disease penetration; the age, health and sex of the subject; the route of administration; and whether the administration is regional or systemic. For example, an effective amount of said compound can be based on the approximate weight of a tumor mass to be treated. The approximate weight of a tumor mass can be determined by calculating the approximate volume of the mass, wherein one cubic centimeter of volume is roughly equivalent to one gram. An effective amount of the compound based on the weight of a tumor mass can be at least about 10 micrograms/gram of tumor mass, and is preferably between about 10-500 micrograms/gram of tumor mass. More preferably, the effective amount is at least about 60 micrograms/gram of tumor mass. Particularly preferably, the effective amount is at least about 100 micrograms/gram of tumor mass. It is preferred that an effective amount based on the weight of the tumor mass be injected directly into the tumor. An effective amount of said compound can also be based on the approximate or estimated body weight of a subject to be treated. Preferably, such effective amounts are administered parenterally or enterally, as described herein. For example, an effective amount of the compound is administered to a subject can range from about 5-3000 micrograms/kg of body weight, and is preferably between about 700-1000 micrograms/kg of body weight, and is more preferably greater than about 1000 micrograms/kg of body weight. One skilled in the art can also readily determine an appropriate dosage regimen for the administration of the compound to a given subject. For example, the compound can be administered to the subject once (e.g., as a single injection or deposition). Alternatively, said compound can be administered once or twice daily to a subject for a period of from about three to about twenty-eight days, more preferably from about seven to about ten days. In a preferred dosage regimen, the compound is administered once a day for seven days. Where a dosage regimen comprises multiple administrations, it is understood that the effective amount of the compound administered to the subject can comprise the total amount of gene product administered over the entire dosage regimen.

The compounds of the presention (e.g. miRNAs or miRNA expression inhibiting compounds) can be administered to a subject by any means suitable for delivering these compounds to cells of the subject. For example, the miRNAs or miR expression inhibiting compounds can be administered by methods suitable to transfect cells of the subject with these compounds, or with nucleic acids comprising sequences encoding these compounds. Preferably, the cells are transfected with a plasmid or viral vector comprising sequences encoding at least one miRNA or miRNA expression inhibiting compound.

Transfection methods for eukaryotic cells are well known in the art, and include, e.g., direct injection of the nucleic acid into the nucleus or pronucleus of a human cell; electroporation; liposome transfer or transfer mediated by lipophilic materials; receptor mediated nucleic acid delivery, bioballistic or particle acceleration; calcium phosphate precipitation, and transfection mediated by viral vectors.

A miRNA or miRNA expression inhibiting compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); periand intra-tissue injection (e.g., peri-tumoral and intra-tumoral injection, intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Preferred administration routes are injection, infusion and direct injection into the tumor.

In the present methods, an miRNA or miRNA expression inhibiting compound can be administered to the subject either as naked RNA, in combination with a delivery reagent, or as a nucleic acid (e.g., a recombinant plasmid or viral vector) comprising sequences that express the miRNA or the miRNA expression inhibiting compound. Suitable delivery reagents include, e.g, the Minis Transit TKO lipophilic reagent; lipofectin; lipofectamine; cellfectin; polycations (e.g., polylysine), and liposomes.

Recombinant plasmids and viral vectors comprising sequences that express the miRNAs or miRNA expression inhibiting compounds, and techniques for delivering such plasmids and vectors to cells, are discussed above.

In a preferred embodiment, liposomes are used to deliver a miRNA or miRNA expression inhibiting compound (or nucleic acids comprising sequences encoding them) to a subject. Liposomes can also increase the blood half-life of the gene products or nucleic acids. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream.

A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference. The liposomes for use in the present methods can comprise a ligand molecule that targets the liposome to cells. Ligands which bind to receptors prevalent in cells, such as monoclonal antibodies that bind to tumor human cell antigens, are preferred. The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference. Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; animated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous wellknown techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive animation using Na(CN)BH3 and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., USA, 18:6949-53. In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the miRNAs or miRNA expression inhibition compounds (or nucleic acids comprising sequences encoding them) to tumor cells.

The miRNAs or miRNA expression inhibition compounds are preferably formulated as pharmaceutical compositions, sometimes called “medicaments,” prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise at least one miRNA or miRNA expression inhibition compound (or at least one nucleic acid comprising sequences encoding them) (e.g., 0.1 to 90% by weight), or a physiologically acceptable salt thereof, mixed with a pharmaceutically-acceptable carrier. The pharmaceutical formulations of the invention can also comprise at least one miRNA or miRNA expression inhibition compound (or at least one nucleic acid comprising sequences encoding them) which are encapsulated by liposomes and a pharmaceutically-acceptable carrier. Preferred pharmaceutically-acceptable carriers are water, buffered water, normal saline, 0.4% saline, 0.3% glycine, hyaluronic acid and the like.

In a particular embodiment, the pharmaceutical compositions of the invention comprise at least one miRNA or miRNA expression inhibition compound (or at least one nucleic acid comprising sequences encoding them) which is resistant to degradation by nucleases. One skilled in the art can readily synthesize nucleic acids which are nuclease resistant, for example by incorporating one or more ribonucleotides that are modified at the 2′-position into the miRNAs. Suitable 2′-modified ribonucleotides include those modified at the 2′-position with fluoro, amino, alkyl, alkoxy, and O-allyl.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceuticallyacceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one miRNA or miRNA expression inhibition compound (or at least one nucleic acid comprising sequences encoding them). A pharmaceutical composition for aerosol (inhalational) administration can comprise 0.0120% by weight, preferably 1%-10% by weight, of the at least one miRNA or miRNA expression inhibition compound (or at least one nucleic acid comprising sequences encoding them) encapsulated in a liposome as described above, and a propellant. A carrier can also be included as desired; e.g., lecithin for intranasal delivery.

The present invention also relates to a method for determining whether a human cell is at risk of a mitochondrial dysfunction comprising a step consisting of determining the expression level of at least one miRNA selected from the group consisting of hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201.

Said method may be thus particularly useful for determining whether a subject is at risk of having a disease associated with a mitochondrial dysfunction. Accordingly a further object of the invention relates to a method for determining whether a subject is at risk of having a disease associated with a mitochondrial dysfunction comprising determining the expression level of at least one miRNa selected from the group consisting of hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201 in a sample obtained from the subject.

Typically the “sample” means any cell or tissue sample derived from the subject. Said sample is obtained for the purpose of the in vitro evaluation. The sample can be fresh, frozen, fixed (e.g., formalin fixed), or embedded (e.g., paraffin embedded). In a particular embodiment the sample results from biopsy performed in the subject. In a particular embodiment the sample can be blood, urine or saliva.

According to the invention, measuring the expression level of the miRNA of the invention in the sample obtained form the patient can be performed by a variety of techniques.

For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted miRNAs is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence based amplification (NASBA).

In a particular embodiment, the determination comprises contacting the sample with selective reagents such as probes or primers and thereby detecting the presence, or measuring the amount of miRNAs originally in the sample. Contacting may be performed in any suitable device, such as a plate, microtiter dish, test tube, well, glass, column, and so forth In specific embodiments, the contacting is performed on a substrate coated with the reagent, such as a miRNA array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a miRNAs hybrid, to be formed between the reagent and the miRNAs of the sample.

Nucleic acids exhibiting sequence complementarity or homology to the miRNAs of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

The probes and primers are “specific” to the miRNAs they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

Accordingly, the present invention concerns the preparation and use of miRNA arrays or miRNA probe arrays, which are macroarrays or microarrays of nucleic acid molecules (probes) that are fully or nearly complementary or identical to a plurality of miRNA molecules positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like.

After an array or a set of miRNA probes is prepared and/or the miRNA in the sample or miRNA probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001). Of particular interest in many embodiments is the use of stringent conditions during hybridization. Stringent conditions are known to those of skill in the art.

Alternatively, miRNAs quantification method may be performed by using stem-loop primers for reverse transcription (RT) followed by a real-time TaqMan® probe. Typically, said method comprises a first step wherein the stem-loop primers are annealed to miRNA targets and extended in the presence of reverse transcriptase. Then miRNA-specific forward primer, TaqMan® probe, and reverse primer are used for PCR reactions. Quantitation of miRNAs is estimated based on measured CT values.

Many miRNA quantification assays are commercially available from Qiagen (S. A. Courtaboeuf, France) or Applied Biosystems (Foster City, USA).

Expression level of a miRNA may be expressed as absolute expression level or normalized expression level. Typically, expression levels are normalized by correcting the absolute expression level of a miRNA by comparing its expression to the expression of a mRNA that is not a relevant for determining the outcome of the atherosclerosis in the patient, e.g., a housekeeping mRNA that is constitutively expressed. Suitable mRNA for normalization include housekeeping mRNAs such as the U6, U24, U48 and S18. This normalization allows the comparison of the expression level in one sample, e.g., a patient sample, to another sample, or between samples from different sources.

Therefore the methods of the invention further comprises a step consisting of comparing the expression level of at least one miRNA determined in the sample of the patient with a reference expression level, wherein a difference between said expression levels is indicative whether the subject is at risk of having a disease associated with a mitochondrial dysfunction.

The invention will be further illustrated by the following figures and examples. However, these examples and figures should not be interpreted in any way as limiting the scope of the present invention.

### EXAMPLE

### Material & Methods

Genomic and Protein Sequences

NCBI entries of the protein sequences of human AGO2 (also referred to as EIF2C2) were systematically retrieved (Gene Identity: 6468775, 14043279, 62913977, 119612613, 29171734, 257467482, 133777965 and 119612614). The dataset for miRNA sequences were downloaded from the Sanger miRBase database Release 13.0 [55]. The studied mitochondrial genomic DNA (mtDNA) sequence was retrieved from NCBI (NC_{—}012920).

Genomic and Protein Bioinformatic Analyses

Genomic and chromosomal locations were analyzed through the Ensembl and UCSC genome browsers (release 59), and our in-house developed bioinformatic tool to question miRNAs and genetic loci/diseases (http://www.mirifix.com, Henrion-Caude personal communication). Mapping of the mature miRNA sequences on genomic nuclear and mitochondrial DNA was performed using BLAT [56]. Conservation of each miRNAs was assessed through BLASTN with default parameter values [55].

Molecular weight of each AGO2 entry was calculated using the ProtParam tool[57] to identify the adequate protein sequences to be further analyzed. Predictions of subcellular localization were done using the following three network-based approaches, namely TargetP 1.1 [29], MitoPROT II v. 1.101 [30], PREDOTAR 1.03 [31], and a support vector machine-based method integrative of multiple features of the protein: physicochemical properties, amino acid compostion, dipeptide compostion of proteins and PSI-BLAST information [32].

Cell Culture

HeLa cells were grown in DMEM medium completed with 10% fetal bovine serum and 100 U/ml penicillin-streptomycin. Similar conditions of culture were used for HEK293 and U2OS cells. Cell lines were purchased from ATCC (ATCC, Manassas, Va., USA). For cell fractionation, HeLa cells were allowed to reach 80-100% confluence. For immunocytochemistry, cells were allowed to reach 60% on glass coverslips.

Isolation of Mitochondria and Cytosol

We isolated mitochondria with the Mitochondria Isolation kit (Miltenyi Biotec, Bergisch Gladbach, Germany) as described [25], with several modifications that allowed the isolation of the cytosol from the same cells. Briefly, HeLa cells were harvested at 80-100% confluency and washed twice with phosphate-buffered saline (PBS). Cells (3×10⁷) were lysed in 1 ml of Lysis Buffer from the kit, complemented with Complete Protease Inhibitor Cocktail Tablets (Roche, Mannheim, Germany). Then, the lysate was divided into two aliquots. In order to isolate mitochondria, the first aliquot was homogenized by shearing through a 29 G needle 55 times. To magnetically label mitochondria, the cell lysate was incubated with 50 μl of monoclonal anti-TOM22-conjugated microBeads for 1 hour at 4° C. Then, the suspension of labeled mitochondria was loaded onto a pre-equilibrated MACS Column (Miltenyi Biotec, Bergisch Gladbach, Germany), previously placed in the magnetic field of a MACS Separator (Miltenyi Biotec, Bergisch Gladbach, Germany). Due to the strong magnetic field generated by the MACS Separator, labeled-mitochondria were retained into the column. The column was washed three times with 3 ml of PEB buffer (PBS pH 7.2, 2 mM EDTA and 0.5% BSA). Subsequently, the column was removed from the magnetic field and the retained mitochondria were eluted in 1.5 ml of PEB buffer. After elution, mitochondria were suspended again in 9 ml of PEB buffer, applied to a fresh column, washed and eluted for a second time. When noted, mitochondria were treated with RNase A. Briefly, isolated mitochondria were resuspended in suspension buffer (0.25 M sucrose, 2 mM MgCl2, 10 mM Tris HCl pH 7.4) and treated with 50 μg protease-free RNase A (USB-Amersham) either for 15 min at room temperature as described in [58], or for 30 min at 4° C. as described in [23] and washed twice in suspension buffer. Mitochondrial pellet was recovered after centrifugation at 13,000×g for 2 min at 4° C. Mitochondrial integrity was monitored after resuspension of the mitochondrial pellet by measuring citrate synthase activity, before and after membrane disruption by adding Triton X-100, as previously described [59].

Mitochondrial fraction of U2OS was isolated using standard method (protocol Mitosciences, adapted from [60]). Extraction of mitochondrial soluble proteins from integral membrane proteins was performed through sodium carbonate treatment as described [61].

In order to isolate the cytosolic fraction, the second aliquot of crude HeLa lysate was homogenized through a glass homogenizer in homogenization buffer (210 mM manitol, 70 mM sucrose, 1 mM EDTA, 10 mM Hepes-NaOH, pH 7.5), as previously described [62]. The homogenate was centrifuged at 2,000×g for 30 min at 4° C. to remove nuclei and unbroken cells. The supernatant was recovered and subsequently centrifuged at 13,000×g for 10 min to give the cytosolic fraction, which was subjected to RNA extraction.

Protein and RNA Extraction

For total protein extraction, HeLa cells were lysed as previously described [33]. Pelleted mitochondria were lysed as described previously [63]. For RNA extraction, pelleted mitochondria and cytosolic fractions were subjected to RNA isolation by Trizol and Trizol LS (Life Technologies, Carlsbad, USA) extraction methods, respectively, according to instructions of the manufacturer.

Microarray miRNA Profiling

MicroRNA microarray analysis was performed by Miltenyi Biotec Company (Milteny Biotec, Bergisch Gladbach, Germany). Briefly, the concentration of mitochondrial and cytosolic RNAs was measured by spectrophotometry at A260/280 and the quality of the RNA sample was assessed using the Agilent 2100 Bioanalyzer (Agilent Technologies, Palo Alto, USA). 2 μg of respective mitochondrial and cytosolic RNA were each mixed with 2.5 fmol of miRControl 3, which comprises 18 RNA oligonucleotides, used as calibrators for normalization. Then mitochondrial RNA was Hy5-labeled while cytosolic RNA was Hy3-labeled using a commercial kit (miRCury™ LNA microRNA Array Power labeling kit, Exiqon, Copenhagen, Denmark). The corresponding total RNA samples were hybridized overnight in a dual colour approach to miRXplore™ Microarrays using the a-Hyb™ Hybridization Station (Miltenyi Biotec, Bergisch Gladbach, Germany). The miRXplore™ Microarray is composed of 6228 DNA oligonucleotides with a sequence being reverse complement to the respective mature miRNA or control RNA, representing a total of 1460 miRNA sequences and 97 controls with all probes spotted in quadruplicates. The 1460 miRNA sequences covered sequence verified miRNAs of the species human (878) mouse (696) and rat (426) as well as viral miRNAs (141) and further mammalian species (76) as deposited in the miRBase sequence database version 13.0. Image capture of microarrays was done with the Agilent's Microarray Scanner System (Agilent Technologies, Palo Alto, USA). Signal quantification of the scanned microarrays was done using ImaGene software Version 8.0 (BioDiscovery, Los Angeles, USA). The data generated for each sample on the array were analyzed with PIQOR Analyzer software. Local background was subtracted from the signal to obtain the net signal intensity and the Hy5/Hy3 ratios. Subsequently, the mean of the ratios of 4 corresponding spots representing the same miRNA was computed. The mean ratios were normalised using the miRControl 3.

Primers, Reverse Transcription and PCR Experiments

For miRNAs, primers were designed as the exact mature miRNA sequences (as indicated at the Sanger miRBase database, v13.0), in comparison to the 16S rRNA. Oligonucleotides for GAPDH were designed through the Primer3 Tool[64], and used at 60° C. for annealing. Primer sequences for COX3 and cyt b were designed as described [65], respectively used at 48° C. and 58° C. for annealing. Each oligonucleotide was applied at a final concentration of 0.4 μM. Reverse-transcription of 1 μg of mitochondrial and cytosolic RNA after treatment with the RQ1 RNase-free DNase I (Promega, Madison, USA) was performed using the miScript Reverse transcriptase kit (Qiagen, Weiden, Germany) according to manufacturers' instructions. PCR were performed in triplicate in 20 μl final volume. Specificity of each PCR products was ensured by the results of melting curve analysis and agarose gel electrophoresis. Separated products were quantitated by band densities using the ImageJ software and normalized to ethidium bromide staining.

Antibodies, Immunoblotting and Immunocytochemistry

The following primary antibodies were commercially purchased: mouse monoclonal anti-ATP5A1, MS507, Mitosciences, Eugene, USA, 1:1000 for immunoblotting and 1:750 for immunofluorescence; mouse monoclonal anti-NDUFA9, MS111, Mitosciences, Eugene, USA, 1:5000; mouse monoclonal anti-VDAC1, MSA03, Mitosciences, Eugene, USA, 1:2500; mouse monoclonal anti-CDK2, sc-6248, Santa Cruz Biotechnology, Santa Cruz, USA, 1:1000; mouse monoclonal anti-actin, VMA1501R, AbCys, Paris, France, 1:500; rabbit monoclonal anti-cythocrome c, 1896-1, Epitomics, Burlingame, USA, 1:2000 and rabbit polyclonal anti-SLUG, sc-15391X, Santa Cruz Biotechnology, Santa Cruz, USA, 1:250. Rabbit polyclonal anti-AGO2 was kindly provided by Pr Tom Hobman (clones 7C6 and 7C3) and the antibody was used at a dilution of 1:1000 for immunoblotting and 1:250 for immunofluorescence and co-immunoprecipitation. Mouse monoclonal anti-AGO2 was kindly provided by Pr Haruhiko Siomi and Pr Mikiko C. Siomi (clone 4G8) and the antibody was used at a dilution of 1:50 for immunofluorescence and at a dilution of 1:300 for Western blot. Secondary antibodies HRP-conjugated anti-mouse and anti-rabbit were purchased (GE Healthcare Bio-Sciences, Little Chalfont, UK). Fluorescein isothiocyanate (FITC)- and tetramethylrhodamine isothiocyanate (TRITC)-labeled secondary antibodies for immunofluorescence microscopy were from Invitrogen (Life Technologies, Carlsbad, USA). Equal amounts of protein (40 μg) were size-separated through a 3-8% sodium dodecyl sulfate-polyacrylamide gel electrophoresis (NuPage Novex Mini gel; Life Technologies, Carlsbad, USA) and were transferred to PVDF membranes (Bio-Rad, Hercules, USA). Membranes were blocked with 4% fat-free milk diluted in PBST (0.05% Tween-20, 1×PBS) for 1 hour at room temperature and incubated with primary antibodies. Antigen-antibody complexes were detected by incubating the membrane with the appropriate secondary antibodies at room temperature for 1 hour. Immunoreactive proteins were visualized with enhanced chemiluminescence and exposed to autoradiographic film (Amersham, GE Healthcare Bio-Sciences, Little Chalfont, UK). Immunocytochemical experiments were performed in HeLa, HEK293 and U2OS cells (American Type Culture Collection (ATCC): CCL-2, Manassas, Va.). Mitochondria were stained with Mitotracker Red CMXRos (Molecular Probes; Life Technologies, Carlsbad, USA) before fixation according to manufacturer's instructions. In all experiments cells were fixed with 4% PFA or 2% formaldehyde for 20 min at room temperature. Cells were then washed three times with PBS, incubated with primary antibodies (1 hour at room temperature), washed, incubated with appropriate secondary antibodies (1 hour at room temperature), washed and mounted with Prolong Gold Antifade medium with Dapi (Life Technologies, Carlsbad, USA). In all experiments, the following controls were included: (i) to assess possible autofluorescence of the samples, the primary and secondary antibodies were omitted in the incubation steps; (ii) to assess possible autofluorescence of secondary antibodies, the primary antibodies were omitted in incubation step. Images were acquired using a confocal microscope Leica SP5. Co-localization of AGO2 with mitochondria was assessed by statistical analysis of the correlation of the intensity values of green (AGO2) and red (mitochondria) pixels in dual-channel images. The JACoP plug-in from ImageJ software was used to calculate the Pearson's correlation coefficient (rp) and to perform cross-correlation analysis (Van Steensel's cross-correlation function, CCF) [28,66,67]. The Pearson's correlation coefficient (rp) describes the correlation of the distributions of signal intensity of pixels between green and red channels [60]. It lies between +1 and −1. From 0 to +1, values indicate significant correlation of green and red images, with 1 indicating 100% co-localization. In the Van Steensel's approach, the CCF is calculated as the value of rp while operating a shift of one of the images relative to the other, then plotting the retrieved rp as a function of the displacement. Co-localization is identified by a Gaussian distribution, while in the opposite situation, the curve appears as a hollow.

Co-Immunoprecipitation of Mitochondrial Transcripts

Co-immunoprecipitation of AGO2 with associated transcripts was performed using μMACS protein A microbeads (MiltenyiBiotec, Bergisch Gladbach, Germany) according to manufacturer's instructions with the following modifications. Briefly, proteins were incubated with protein A microbeads and either rabbit polyclonal anti-AGO2 (clone 7C6 or 7C3) or rabbit polyclonal anti-SLUG as a negative control (sc-15391X, Santa Cruz Biotechnology, Santa Cruz, USA) for 1 hour at 4° C. on ice. After 6 washes of varying stringency onto microcolumns, proteins were treated with proteinase K in washing buffer complemented with 0.1% SDS, 25 mM EDTA for 30′ at 42° C. Transcripts were then eluted at 80° C. with pre-heated RNase-free water. Subsequently, RNA was extracted with Trizol reagent (Lifetechnologies, Carlsbad, USA) (according to manufacturer's protocol), treated with the RQ1 RNase-free DNase I (Promega, Madison, USA) (according to manufacturer's instructions), reverse transcribed with the GeneAmp RNA PCR kit (Applied Biosystems, Life Technologies, Carlsbad, USA) (according to manufacturers' instructions), and subsequently amplified by PCR using standard conditions.

Structural Analyses of miRNAs

The minimal folding energy (MFE), expressed in kcal/mol, is a method of calculating the thermodynamic stability of the secondary structure of RNA [68]. The lower the MFE of a molecule, the more stable the secondary structure. Minimal folding free energies (MFEs) of pre-miRNAs were estimated using the program RNAfold (Vienna RNA Package, version 2.0.0) with default parameter values [69,70]. Because MFE values are strongly correlated with the length of the sequence we normalized the MFE by calculating the adjusted MFE (AMFE) using the following equation: AMFE=[(−MFE/length of RNA sequence)×100] [71]. MFE index (MFEI) was calculated using the following equation: MFEI=AMFE/(G+C)% [71].

Computational Prediction of miRNA Targets

For each miRNA, genome-wide miRNA nuclear targets were determined using miRDB as a tool of primary focus on mature miRNAs, which are the functional carriers of miRNA-mediated regulation of gene expression [72]. Enrichment in mitochondrial protein-coding gene was identified from the overlap between predicted targets and mitochondrial proteome [40]. To scan the mitochondrial genome for potential miRNA target sites, we used four independent algorithms RNA22, Target Scan, RegRNA and miRWalk. RegRNA is based on the miRanda algorithm, which relies on both the complementarity of miRNA and target sequences and on the conservation of the target site [73]. Target Scan algorithm (http://www.targetscan.org/) searches for the presence of conserved target sites that match the seed region of each miRNA and assesses the structural accessibility of the predicted target site. RNA22 is based on the Teiresias algorithm, which relies on a pattern-based approach without using conservation filters [74]. The miRWalk algorithm is based on a computational approach starting with a heptamer seed of miRNA and identifies possible complementary on the complete mitochondrial genome (Ruprecht-Karls-Universität Heidelberg, Medizinische Fakultät Mannheim, Germany). A probability distribution of random matches of a subsequence (miRNA 5′ end sequence) in the given sequence was calculated by using Poisson distribution where a low probability implies a significant hit. All predictions algorithms were run under default parameters.

Expression Data and Pathway Database Analysis

All data is MIAME compliant and the data presented in this manuscript have been deposited in NCBI's Gene Expression Omnibus. miRNA expression profiling data was accessed through mimiRNA [36] and MirZ [35]. The ExParser algorithm [36] was used to compile datasets of gene that multiple experimental sources classified as targets of one mitomiR and/or as genes co-regulated with the mitomiR. The datasets obtained for each miRNA were uploaded into MetaCore□, a systems biology pathway analysis tool[41]. Ontology enrichment analysis was performed using general enrichment categories, i.e. Gene Ontology and GeneGo□ Processes, which represents prebuilt networks of manually curated protein-protein, or protein-nucleic acid interactions, assembled on the basis of proven literature evidence. The enrichment calculation uses the Fisher exact test or hypergeometric distribution to calculate the probability that the degree of overlap between the list of miRNA targets (generated from the ExParser query), and the protein represented in the functional ontology category can happen by chance, given an identical number of proteins selected at random from the protein universe annotated within the ontology. The p-value generated is used to rank the functional representation of the miRNA targets in each ontology by their significance to the list of targets, thereby identifying biological process likely to be affected. Probabilities were calculated according to the manufacturer's recommendations.

Statistics and Microarray Analyses

Statistical significance was validated using a two-tailed Student's t-test assuming unequal variance, respectively a Fisher-Snedecor F-test for variance distribution whereby significance was achieved for p<0.05 in each test. Data analysis of the microarray was performed as follows: first, scan images of the microarrays were analyzed using the ImaGene Software (Biodiscovery). During the image analysis, irregular spots, dust particles or areas of high background were discarded, and exact position of each spot, its identity, its signal intensity, and surrounding background are saved. Second, primary data analysis was then performed calculating the net signal intensities (spot signal intensity minus background signal intensity). As filter criteria, we applied the 50% percentile of the background signal intensities. For calculation of the Hy5/Hy3 ratio, only spots/genes with signal equal or higher than the 50% percentile of the background signal intensities were taken into account. For each microarray this Hy5 and Hy3 default/threshold value was adjusted by the median of the calibrators. Third, data were normalized to correct for dye bias such as inconsistent labelling efficiencies, varying quantum yields of the dyes, or different scanning parameters. To overcome these systematic variations a normalization using spiked calibration controls was performed. After normalization, ratios of the mitochondrial vs. cytosol for each spot were calculated, and subsequently the mean of the ratio from 4 spots was calculated. Ratios were calculated for values above the adjusted threshold value (calculated based on the 50% percentile of the background signal intensities and adjusted by the mean of the calibration oligos): avg(Hy5-bkg)<adjust Hy5 default AND avg(Hy3-bkg)<adjust Hy3 default.

Results

Investigating AGO2 at the Mitochondria

We first addressed the possibility that endogenous AGO2 could localize to mitochondria. To isolate highly-purified mitochondria, we performed cell fractionation combined with subsequent immunoisolation of mitochondria. This isolation procedure adapted from Hornig-Do et al. [25] was checked for efficiency by measuring the activity of mitochondrial and cytosolic marker enzymes. Mitochondrial fraction was analyzed for its purity by immunoblot as assessed by the mitochondrial marker ATP synthase subunit a 1 (ATP5A1) and the nuclear/cytosolic marker cyclin-dependent kinase 2 (CDK2), which indicated the lack of nuclear and cytosolic contaminants reproducibly. To study the localization of endogenous AGO2, we chose the same polyclonal antibody as that used in the founding studies of AGO2 localization [16,26,27]. By immunoblotting, we detected AGO2 as a band of ˜102 kDa in HeLa mitochondrial proteins as well as in the total protein extract from the same cells. Assessment of actin as a cytosolic marker further validated the lack of cytosolic contaminants. To further ascertain AGO2 at mitochondria, we extracted the crude membrane pellet and the soluble protein fraction from mitochondria isolated from a U2OS cell line. Mitochondria were incubated in hypo-osmotic buffer alone or supplemented with either 1 M NaCl or 0.1 M Na₂CO₃, pH 11 prior and after fragilization. Efficacy of treatments was ensured by immunoblotting detection of the following proteins: voltage-dependent anion channel 1 (VDAC1), cytochrome c (CYCS) and NADH dehydrogenase (ubiquinone) 1 a subcomplex 9 (NDUFA9), which all showed patterns consistent with the known location of those markers, respectively as a mitochondrial membrane marker, an intermembrane marker and a mitochondrial membrane marker. AGO2 was detected in the mitochondrial membrane fraction and to a lesser extent in the soluble fraction, suggesting its preferential association to mitochondrial membranes.

Mitochondrial localization of AGO2 was further assessed by immunofluorescence confocal microscopy. Immunostaining of AGO2 showed a consistent punctuated cytoplasmic and nuclear pattern. Overlay of AGO2 immunostaining with mitochondrial staining indicated a partial co-localization as assessed by two distinct antibodies. Correlation between the intensities of green (AGO2) and red (mitochondria) in dual-channel images was studied using the Pearson's correlation coefficient (rp) in three different human cell lines, whether tumoral or transformed i.e HeLa, U2OS and HEK293 cells. Average values of rp>0.5 indicated a significant correlation of green and red pixels, which was consistent in all cell types. Evidence of co-localization was improved using an additional parameter, which is the Van Steensel's cross-correlation function (CCF; [28]). Plotted CCF revealed curves with a bell-like shape further indicating that AGO2 and mitochondria were positively correlated.

Interestingly, the use of four prediction programs to identify subcellular protein localization (i.e TargetP [29], MitoProt II [30], Predotar [31] and ESLPred [32]) all consistently predicted a mitochondrial localization of AGO2, specifically when assessing the CRA-b isoform. For that isoform, TargetP and MITOProt II delineated an N-terminal region of 9-24 amino acids that could support a mitochondrial targeting sequence.

To assess the possibility of AGO2 to function at the mitochondria, we then examined whether AGO2 could interact with the mitochondrial transcript cytochrome c oxidase III (COX3) as previously found in HEK293 cells [33]. By co-immunoprecipitating endogenous AGO2 with the associated RNAs in HeLa cell extracts and subsequent RT-PCR, we consistently identified COX3 as reproducibly associated, in comparison to a mitochondrial transcript cytochrome b (cyt b) and a cytosolic transcript glyceraldehyde-3-phosphate dehydrogenase (GAPDH) that were not associated. Those co-immunoprecipitation results were performed along the control immunoprecipitation of a transcriptional factor SLUG, which as expected did not lead to the identification of any targets. Collectively, our data support the novel localization and functioning of AGO2 at the human mitochondria, a finding that prompted us to search for mitochondrial miRNAs.

Isolation of Mitochondrial and Cytosolic RNAs

We reasoned that differential identification of miRNAs in subcellular compartments from the same cells would provide the most reliable and effective method to investigate localization of miRNAs to mitochondria. Our experimental design enabled us to isolate mitochondria and cytosol fractions from the same cells and to profile differentially expressed miRNAs in each fraction using the miRXplore™ microarrays. Total RNA was isolated respectively from mitochondrial and cytosolic fractions. Each RNA fraction was examined for its integrity, quality and purity through the Agilent 2100 Bioanalyzer. Electrophoretic gel images were observed for mitochondrial and cytosolic RNA fractions. Consistently with the electrophoregrams, the 28S rRNA and 18S rRNA, which are located exclusively in the cytoplasm, were not observed in the mitochondrial RNA fraction indicating that mitochondrial and cytoplasmic RNA fractions were distinct. We further applied the RNA Integrity Number (RIN) algorithm to each sample to assign an integrity value [34]. Mean RIN values were respectively of 2.8 and 8.3 for the mitochondrial and cytosolic fractions. The cytosolic value was indicative of RNA of high quality. Since no standard exists as to the mitochondrial fraction, which consists of a distinct RNA population, we considered a value smaller than 6 as an indication of the depletion of cytosolic RNAs that we further ascertained by the depletion of cytosolic 18S and 28S rRNAs from mitochondrial RNA in the Bioanalyzer run. Finally, we assessed the purity of mitochondrial and cytosolic RNA fractions by reverse transcription-polymerase chain reaction (RT-PCR). 16S rRNA, which was chosen as a mitochondrial marker and thereby as a positive control for mitochondrial RNA, was enriched in the mitochondrial RNA fraction whereas it was depleted from the cytosolic RNA. In contrast, GAPDH, an unambiguous cytosolic marker, could be amplified exclusively in the cytosolic fraction. Altogether, these data indicated that no cytosolic contaminants could be detected in mitochondrial RNA, while faint signals of mitochondrial RNA were detected in the cytosol. These results are consistent with a high level of purity in either fraction.

Differential Expression of Mature miRNAs in the Mitochondria and the Cytosol

We profiled miRNAs at the genome-wide scale in the mitochondrial and cytosolic RNA fractions purified from HeLa cells. Mitochondrial and cytosolic RNA were labeled using the fluorescent dyes Hy5 and Hy3, respectively, and hybridized to microarrays in three independent analyses. miRNAs showing significant hybridization signals were analyzed for their enrichment either in the mitochondrial or the cytosolic RNA fractions by determining the Hy5/Hy3 ratio values. Using a cutoff fold of enrichment of 1.5, we identified a subset of 57 miRNAs differentially expressed in the mitochondrial and cytosolic RNA fractions. Two subgroups were clearly identified suggesting that a specific population of miRNAs was likely compartmentalized in mitochondria. While 44 miRNAs showed a greater enrichment in the cytosolic Hy3-labeled RNA fraction, 13 miRNAs were significantly and reproducibly enriched in the mitochondrial Hy5-labeled RNA sample (ranging from 1.5- to 56-fold), namely hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201. In parallel, microarray experiments were repeated thrice with RNase A-treated mitochondria, giving a consistent signature of the same mitochondrial-enriched miRNAs. This latter result emphasized the actual localization of those miRNAs within the mitochondria. The data are accessible through GEO Series (http://www.ncbi.nlm.nih.gov/geo/query/acc.cgi?acc=GSE24761).

Microarray data was independently verified by RT-PCR analysis assessing hsa-miR-494, hsa-miR-1275 and hsa-miR-1974. For each we assessed the differential expression in HeLa mitochondria relative to the mitochondrial control, 16S rRNA. All miRNAs were significantly enriched in mitochondria as compared to the cytosol (p<0.03). Finally, we further validated our results through a systematic comparison with previously released miRNA expression data in HeLa cells, using total RNA [35,36]. We observed that the vast majority of cytosolic-enriched miRNAs (84%) were reproducibly identified in previous HeLa expression data while most of the mitochondrial-enriched miRNAs (69%) were absent as expected from their dilution in total RNA. Thus, our findings were consistent with a signature of 13 miRNAs significantly enriched in the mitochondrial RNA fraction. Notably, three of those miRNAs, namely hsa-mir-1974, hsa-mir-1977 and hsa-mir-1978 are non-canonical miRNAs for they map to the mitochondrial genome, and they map to tRNA and rRNA genes. However, their size, which ranges from 21-23 nucleotides is similar to miRNAs. The identification of several small RNA similar in size to miRNAs that are derived from abundant non-coding RNAs [37,38] prompted us to consider hsa-mir-1974, hsa-mir-1977 and hsa-mir-1978 as potential components of RNAi at mitochondria. For simplicity, we termed those three mlRNA-like RNAs and the other detected miRNAs, ‘mitomiRs’ in reference to their preferential localization in HeLa mitochondria. We examined the conservation of all mitomiRs by systematic BLAST of their sequences against those from metazoan species. To our surprise, despite the fact that most miRNAs are conserved accross metazoans [39], only two of the 13 mitomiRs, i.e hsa-miR-494 and hsa-miR-328, were highly conserved while the others were either human-specific or only conserved in primates (Table 1).

MitomiRs Targeting Analyses

We systematically assessed the computational targeting of all mitomiRs. To obtain unbiased predictions, we analyzed the targeting of the 13 mitomiRs in parallel to a control sample of 13 cytosolic-enriched miRNAs. For each miRNA, targets were predicted and then analyzed for enrichment in nuclear genes coding for proteins known as mitochondrial[40]. Mean percentage of these genes within the mitomiR target set reached 17±3 versus 17.6±2.8 in the control set. This difference was not statistically significant. Thus, mitomiRs appeared to lack any preferential predicted targeting of the mitochondrial genes encoded by the nuclear genome.

We then questioned the functional relevance of those mitomiRs. To this end, we used the ExParser algorithm with the publicly available datasets to retrieve the mRNA genes experimentally classified as targets of miRNAs and/or as co-regulated in their expression with mitomiRs [36]. While lack of available experimental data precluded systematic questioning, we were able to analyze the target and/or co-regulated mRNAs for hsa-miR-328, hsa-miR-494, hsa-miR-513 and hsa-miR-638. For each mitomiR, the compiled mRNAs were then assessed for their biological significance using a systems biology pathway analysis tool [41]. We found that all four mitomiRs were significantly involved in mitochondrial homeostasis, e.g hsa-mir-494 and hsa-mir-513 are both involved in ATP synthesis coupled electron transport. One common feature of their involvement pointed out their role in translation initiation and in cell cycle. In particular, the most significant result of hsa-mir-494 is in mitochondrial translation (p-value=3.5×10⁻⁷).

Subsequently, we hypothesized that mitomiRs may directly affect the mitochondrial genome. To scan the mitochondrial genome for potential miRNA target sites, we used four independent and complementary algorithms RNA22, RegRNA, miRWalk and Target Scan (described in the Methods). Ten of the 13 mitomiRs were predicted to target a total of 120 target sites along the mtDNA sequence. We found target sites mapping each mtDNA-encoded protein genes, except for ND4L (Table 2). Merging the targeting analyses from the independent searches highlighted that the most frequent target sites were located at ND1, ND4, ND5, ND6, COX1 and COX2 (Table 2). Interestingly, the first four genes encode the components of the first complex of the respiratory chain. However, it should be emphasized that the actual location of targets does not necessarily concern the targeted gene given the polycistronic transcription of mitochondrial genome (reviewed in [42]). To gain insight into the biological relevance of those predictions, we performed the same analysis with the control set of cytosol-enriched miRNAs, and found a preferential targeting of mitomiRs versus cytosolic miRNAs.

Genomics and Intrinsic Features of the mitomiRs

To gain insights into the molecular basis underlying the mitochondrial localization of miRNAs, we first questioned their genomics. Since it is widely accepted that most miRNAs share regulatory elements with their genomic environment, and when intragenic are typically co-processed from the host gene mRNAs, we inferred that the genomic location of mitomiRs might be informative [43]. Interestingly, the 13 mature mitomiRs appear to be expressed from 15 miRNA genes (Table 3). Of those 15 genes, 9 were intragenic while 6 were intergenic (Table 3). We found that genomic locations of the mitomiRs, besides hsa-miR-513a and hsa-miR-1275 were all relevant to mitochondria. In particular, of 9 intragenic mitomiRs, 4 were hosted in mitochondrial genes (Table 3). Strikingly, hsa-miR-1974, hsa-miR-1977 and hsa-miR-1978 also exhibited a perfect match in the mitochondrial genome with two mitochondrial tRNA genes, TRNE and TRNN and with a stretch of the mitochondrial rRNA sequence RNRJ, respectively. Thus, in addition to the nuclear transcription that can be assumed from the detection of hsa-miR-1974 in the cytosol, it will remain to be ascertained whether the transcription of those 3 mitomiRs could also occur from the mitochondrial genome.

Secondly, we systematically analyzed the intrinsic features of mitomiRs. We assessed their lengths and thermodynamic features in comparison to the same control sample of 13 cytosolic-enriched miRNAs. Unequivocally, the control sample shared all expected features of miRNAs, in particular an average length of 22-nt for the mature and of 82-nt for the pre-miRNA sequence. In contrast, the length of the mature mitomiRs varied substantially. Three mitomiRs were smaller than 19 nt. In fact, the length distribution of the mature mitomiRs was significantly different from the control (p<0.005) but did not correlate with a difference at the pre-miRNA sequence level, which remained comparable (p=0.2). Thus, we assessed the thermodynamic stability of the secondary structures of the mitomiRs by calculating their minimum folding energy (MFE). MFE displayed a significantly different distribution than the control group (p=0.01). Because MFE is strongly correlated with the length of the sequence, we also calculated the adjusted MFE (AMFE). Again, the distribution of AMFE values was significantly different from that of the control miRNAs (p<0.05), which revealed that mitochondrial-enriched miRNAs shared specific distinctive features as a group.

Altogether, our findings illustrate a species-specific signature of mitochondrial miRNAs of unusual sizing that exhibit unique thermodynamic features. These results suggest a set of emerging features of structural properties that should help in the identification of other mitomiRs.

### DISCUSSION

Little is known about the crosstalk between the nucleus and mitochondria, despite the fact that this communication has great relevance for understanding integrative cellular signaling pathways. In this report, we have used complementary approaches to provide evidence that all components of RNA interference are present at the mitochondria in human cells.

We identified a set of 13 miRNAs significantly enriched in mitochondria purified from HeLa cells that we referred to as mitomiRs. We believe that our experimental design, focusing on the differential expression of miRNAs in subcellular fractions isolated from the same cells, favored the identification of a specific signature of miRNAs in mitochondria. Recently, comparable approaches were successfully used for the identification of nuclear and nucleolar small RNAs [19,44]. In our methodology, the choice of immunoisolating mitochondria after differential centrifugation gave us the additional opportunity to wash the organelles in stringent conditions, leading to highly purified mitochondrial fractions. Our findings not only identify a novel set of mitochondrial miRNAs in humans, but also confirm the previous finding of enrichment of miR-494 in rat liver mitochondria [23]. Our analysis of genes hsa-mir-494 functionally-related revealed its involvement in regulating translation in mitochondria. Thus beyond a role in the cytosol, hsa-miR-494 likely displays conserved functions relevant to its mitochondrial localization.

Our study further introduces the idea that a nuclear outsourcing of miRNAs to mitochondria is conserved in mammals. Indeed, extensive mapping of our mitomiRs, as well as the mitochondrial miRNAs identified in rat liver [23] (data not shown) demonstrated that they mostly originated from the nucleus. Noteworthy, hsa-miR-1974, hsa-miR-1977 and hsa-miR-1978 are considered non-canonical miRNAs because they map to mitochondrial tRNA and rRNA genes. At this stage, our data on other mitomiRs and AGO2 at the mitochondria prompt to gain more insights into the accuracy of those three miRNA-like RNA production, whether from nuclear or mitochondrial genome, and from specific enzymatic cleavage or from random degradation. As for hsa-miR-1201, which is also questioned as a genuine miRNA due to the overlap with an annotated small nucleolar RNA (SNORD126), we considered that its binding to both AGO2 and mRNAs [45] sufficiently argued for its possible functioning as a mitomiR. Although the mechanism of miRNA localization to mitochondria is unclear at this stage, our analysis suggests that their hosting in specific genomic regions, together with their lengths and thermodynamics may play a significant role in the specificity of this subcellular localization. Our observation also indicates that most human mitomiRs are not conserved beyond primates, which may suggest a species-specific targeting of miRNAs to human mitochondria. We propose that this biased lack of conservation throughout species likely relates to the peculiar evolutionary pressure that concerns mitochondrial genomes [46,47]. We speculate that further investigation specifically on hsa-miR-1974, hsa-miR-1977 and hsa-miR-1978, which are human-specific only may greatly serve our understanding into both the origin and evolution of miRNAs.

The actual mitochondrial localization of some miRNAs implies that small RNA-mediated processes may regulate mitochondrial biogenesis and function. This hypothesis involving an additional level of nuclear control of mitochondria is emphasized by our finding that AGO2 localizes to mitochondria, and that we and, others, identified its binding to the mitochondrial transcripts COX3 and tRNA^{Met} [24,33]. AGO2 may be considered as a versatile protein, localized to several sites within the cell, including unstructured foci and vesicles [16,17, 21,48], the Golgi apparatus and the endoplasmic reticulum [26] but also the nucleus [20]. Yet, it is not clear which of these cellular structures are necessary for AGO2 functions [49]. Depending on its localization, different functions have already been ascribed to AGO2, such as post-transcriptional gene silencing or reversible translational regulation in P-bodies [50] and stress granules [16], and transcriptional regulation in the nucleus [51]. Given the unique features of mitochondria in terms of genomic organization and regulation [52], it is difficult at this stage to predict role of AGO2 at the mitochondria. Here, our prediction that the isoforms of AGO2 may be differently regulated in respect to their localization to mitochondria adds an additional layer of complexity. Which of the AGO2 isoforms actually localizes to mitochondria, where in the mitochondria and what is the underlying mechanism of such mitochondrial targeting remain to be explored in future studies. In this respect, interesting insights come from the proteomic studies of AGO2 partners, which identified mitochondrial proteins mostly from the inner membrane, including many ATP/ADP translocases, carriers and ribosomal proteins as binding partners [53]. These data, altogether with the recent finding that AGO2 also associates with HSP90 [54] provide interesting rationales as to a possible mechanism for a mitochondrial import of AGO2. Furthermore, our computational identification of miRNA targets in the mitochondrial genome actually provided the first step towards elucidating the functions of AGO2 at the mitochondria. However, technical limitations of directly transfecting mitochondria in vivo will make it challenging to test the classical approaches to characterize the regulatory role of mitomiRs and AGO2 at the mitochondria.

To conclude, the nuclear outsourcing of miRNAs and AGO2 at the mitochondria is likely an additional mechanism mediating the crosstalk between the nucleus and mitochondria. Based on our study, consideration of the mitomiRs as a new species for mitochondrial regulatory RNAs may lead to a deeper understanding of signaling pathways that require nuclear-mitochondrial cooperation. Elucidating the preferential distribution of miRNAs to mitochondria should then provide a first framework to further investigate their organelle-specific functions and to unravel their potential in devising new therapeutic strategies.

TABLE 1
Cross-species conservation of pre-miRNA sequences in metazoans

| miRNA | | Conservation |
|---|---|---|
| identifier | Species (taxonomic orders) | score* |
| hsa-miR-1973 | Hsa (Primates) | 0 |
| hsa-miR-1275 | Hsa, Ptr, Ppy (Primates) | 1 |
| hsa-miR-494 | Hsa, Ptr, Ppy, Mml, (Primates), Mmu, | 2 |
| | Rno (Glires), Cfa (Carnivora), Eca, Bta | |
| | (Perissodactyla), Ssc (Cetertiodactyla) | |
| hsa-miR-513a | Hsa, Ptr, Ppy, Mml, Age, Ssy, Pbi | 1 |
| | (Primates) | |
| hsa-miR-1246 | Hsa, Ptr, Ppy (Primates) | 1 |
| hsa-miR-328 | Hsa, Ptr, Ppy, (Primates), Mmu, | 2 |
| | Rno (Glires), Cfa (Carnivora), Eca, | |
| | Bta (Perissodactyla), Ssc (Cetertiodactyla) | |
| hsa-miR-1908 | Hsa, Ppy (Primates) | 1 |
| hsa-miR-1972 | Hsa (Primates) | 0 |
| hsa-miR-1974 | Hsa (Primates) | 0 |
| hsa-miR-1977 | Hsa (Primates) | 0 |
| hsa-miR-638 | Hsa, Ppy, Mml (Primates) | 1 |
| hsa-miR-1978 | Hsa (Primates) | 0 |
| hsa-miR-1201 | Hsa, Ptr, Ppy (Primates) | 1 |

*Conservation score was assigned as follows: 0 for miRNA sequences that were human specific; 1 for miRNAs sequences conserved in primates; 2 for miRNA sequences conserved in more than 2 orders.

TABLE 2
List of mitomiR-predicted target sites in the mitochondrial genome.

| | Computational tools | | | | |
|---|---|---|---|---|---|
| | RNA22 | | MiRWalk | | Target Scan |
| | Gene hosting | RegRNA | Gene hosting | | Gene hosting |
| | miRNA target | Gene hosting | miRNA target | p-value | miRNA target |
| mitomiR | sites | miRNA target sites | sites | (<0.05) | sites |
| hsa-miR- | — | — | — | | — |
| 1973 | | | | | |
| hsa-miR- | ND5, CYTB, | ND4, ND5, D-loop | ND6 | 0.008 | COX2, ND4, |
| 1275 | COX2, ATP8, | | | | ND6 |
| | ND2, COX1, D- | | | | |
| | loop | | | | |
| hsa-miR- | — | — | — | | — |
| 494 | | | | | |
| hsa-miR- | — | — | — | | — |
| 513a-5p | | | | | |
| hsa-miR- | ND5, COX1 | — | COX1 | 0.002 | ND5, COX1 |
| 1246 | | | | | |
| hsa-miR- | RNR1 | — | — | | — |
| 328-5p | | | | | |
| hsa-miR- | ND6, CYTB, ND4, | COX1, RNR2, D- | COX1 | 0.02 | D-loop, RNR2, |
| 1908 | COX2, ND1 | loop, ND3, TRNN, | | | COX1, ND3 |
| | | ND5, ND4, | | | |
| | | ATP8/ATP6 | | | |
| hsa-miR- | COX1, COX3, L- | COX1 | — | | COX1 |
| 1972 | strand replication | | | | |
| | origin region | | | | |
| hsa-miR- | ND4, TRNN, | TRNN | — | | TRNN, COX1, |
| 1977 | TRNP, ND2, | | | | ND5, CYTB |
| | RNR2, ND5, | | | | |
| | TRNL2 | | | | |
| hsa-miR- | ATP6, TRNP, | COX2 | COX2 | 0.01 | COX2 |
| 638 | TRNY, D-loop, | | | | |
| | COX1, ND5, ND2, | | | | |
| | CYTB, ATP8, | | | | |
| | ND1, ND3, RNR2 | | | | |
| hsa-miR- | ND6/TRNE, ND5, | ND6/TRNE, ND1 | — | | Dloop, ND1, |
| 1974 | CYTB, ND4, | | | | ND2, ND5, |
| | ATP6, | | | | TRNE |
| | TRNS2/TRNL, | | | | |
| | ND1 | | | | |
| hsa-miR- | ND1, COX2, | — | — | | Dloop, RNR2, |
| 1978 | COX1 | | | | ND2, COX1, |
| | | | | | COX2 |
| hsa-miR- | TRNF, COX2, | — | — | | — |
| 1201 | ATP6 | | | | |

— indicates the lack of targeting sites predicted at the level of mitochondrial genome.

The mitochondrial genes indicated in bold present overlapping target sites between RegRNA and RNA22 or RegRNA and miRWalk. The (promoter or mRNA sequence) is calculated by. P-values were calculated using Poisson distribution as a probability distribution of random matches of a subsequence (miRNA 5′ end sequence) in the given sequence calculated. Low probability implies a significant hit (p<0.05).

TABLE 3
Genomic and chromosomal location of mitomiRs

| miRNA (miRBase | | Chromosomal | | |
|---|---|---|---|---|
| v.13) | Genomic location (hg19) | location (hg19) | Host gene | Genomic link to mitochondria - Evidence |
| hsa-miR-1973 | Chr4: 117220905-117220924 (+) | 4q26 | Intergenic | Embedded in a region of mitochondrial pseudogenes. |
| hsa-miR-1275 | Chr6: 33967794-33967810 (−) | 6p21.31 | Intergenic | None. |
| hsa-miR-494 | Chr14: 101496018-101496039 (+) | 14q32.31 | Intergenic | Embedded in a syntenic conserved region of |
| | | | | mitochondrial carrier proteins [75]. |
| hsa-miR-513a | (1) Chr X: 146295056-146295073 | Xq27.3 | Intergenic | Localization (1) and (2): none. |
| | (−) | | | |
| | (2) Chr X: 146307418-146307435 | | | |
| | (−) | | | |
| hsa-miR-1246 | Chr2: 177465752-177465770 (−) | 2q31.1 | Intergenic | Embedded in a region of mitochondrial genes—Locus |
| | | | | involved in mitochondrial disorders (EOMFC). |
| hsa-miR-328 | Chr16: 67236230-67236251 (−) | 16q22.1 | Intragenic | Locus involved in mitochondrial disorders (CDG2H, |
| | | | | MDDS2). |
| hsa-miR-1908 | Chr11: 61582681-61582701 (−) | 11q12.2 | Intragenic | Hosted in FADS1 intron (mitochondrial localization [76]). |
| hsa-miR-1972 | (1) Chr16: 151041224-15104246 | (1) 16q13.11 | Intragenic | Localization (1): None. |
| | (−) | | | |
| | (2) Chr16: 70064295-70064317 | (2) 16q22.1 | | Localization (2): Locus involved in mitochondrial disorder |
| | (+) | | | (CDG2H, MDDS2). |
| hsa-miR-1974 | Chr5: 93905172-93905194 (−) | 5q15 | Intragenic | Hosted in mitochondrial pseudogene (AC093311.4) and |
| | | | | embedded in a region of mitochondrial pseudogenes. |
| | | | | Likely transcription from the mitochondrial genome |
| | | | | (TRNE, M: 14675-14697 (−)). |
| hsa-miR-1977 | Chr1: 566242-566263 (−) | 1p36.33 | Intragenic | Hosted in mitochondrial pseudogene (AC114498.7) and |
| | | | | embedded in a region of mitochondrial pseudogenes. |
| | | | | Likely transcription from the mitochondrial genome |
| | | | | (TRNN, M: 5693-5714 (−)). |
| hsa-miR-638 | Chr19: 10829095-10829119 (+) | 19p13.2 | Intragenic | Hosted in DNM2 (mitochondrial localization [40]). |
| hsa-miR-1978 | Chr2: 149639365-149639385 (−) | 2q23.1 | Intragenic | Possible transcription from the mitochondrial genome |
| | | | | (RNR1, M: 654-674 (+)). |
| hsa-miR-1201 | Chr14: 19864456-19864479 (−) | 14q11.2 | Intragenic | Locus involved in mitochondrial disorders (PCK2D). |

### REFERENCES

Throughout this application, various references describe the state of the art to which this invention pertains. The disclosures of these references are hereby incorporated by reference into the present disclosure.
1. Entelis N S, Kolesnikova O A, Martin R P, Tarassov I A (2001) RNA delivery into mitochondria. Adv Drug Deliv Rev 49: 199-215.
2. O'Brien T W, Denslow N D, Anders J C, Courtney B C (1990) The translation system of mammalian mitochondria. Biochim Biophys Acta 1050: 174-178.
3. Chang D D, Clayton D A (1987) A mammalian mitochondrial RNA processing activity contains nucleus-encoded RNA. Science 235: 1178-1184.
4. Li K, Smagula C S, Parsons W J, Richardson J A, Gonzalez M, et al. (1994) Subcellular partitioning of MRP RNA assessed by ultrastructural and biochemical analysis. J Cell Biol 124: 871-882.
5. Topper J N, Clayton D A (1990) Secondary structure of the RNA component of a nuclear/mitochondrial ribonucleoprotein. J Biol Chem 265: 13254-13262.
6. Magalhaes P J, Andreu A L, Schon E A (1998) Evidence for the presence of 5S rRNA in mammalian mitochondria. Mol Biol Cell 9: 2375-2382.
7. Smirnov A, Tarassov I, Mager-Heckel A M, Letzelter M, Martin R P, et al. (2008) Two distinct structural elements of 5S rRNA are needed for its import into human mitochondria. Rna 14: 749-759.
8. Rubio M A, Rinehart J J, Krett B, Duvezin-Caubet S, Reichert A S, et al. (2008) Mammalian mitochondria have the innate ability to import tRNAs by a mechanism distinct from protein import. Proc Natl Acad Sci USA 105: 9186-9191.
9. Bartel D P (2004) MicroRNAs: genomics, biogenesis, mechanism, and function. Cell 116: 281-297.
10. Kawamata T, Tomari Y Making RISC. Trends Biochem Sci 35: 368-376.
11. Ketting R F The many faces of RNAi. Dev Cell 20: 148-161.
12. Fabian M R, Sonenberg N, Filipowicz W Regulation of mRNA translation and stability by microRNAs. Annu Rev Biochem 79: 351-379.
13. Liu J, Carmell M A, Rivas F V, Marsden C G, Thomson J M, et al. (2004) Argonaute2 is the catalytic engine of mammalian RNAi. Science 305: 1437-1441.
14. Meister G, Landthaler M, Patkaniowska A, Dorsett Y, Teng G, et al. (2004) Human Argonaute2 mediates RNA cleavage targeted by miRNAs and siRNAs. Mol Cell 15: 185-197.
15. Schmitter D, Filkowski J, Sewer A, Pillai R S, Oakeley E J, et al. (2006) Effects of Dicer and Argonaute down-regulation on mRNA levels in human HEK293 cells. Nucleic Acids Res 34: 4801-4815.
16. Leung A K, Calabrese J M, Sharp P A (2006) Quantitative analysis of Argonaute protein reveals microRNA-dependent localization to stress granules. Proc Natl Acad Sci USA 103: 18125-18130.
17. Liu J, Valencia-Sanchez M A, Hannon G J, Parker R (2005) MicroRNA-dependent localization of targeted mRNAs to mammalian P-bodies. Nat Cell Biol 7: 719-723.
18. Hwang H W, Wentzel E A, Mendell J T (2007) A hexanucleotide element directs microRNA nuclear import. Science 315: 97-100.
19. Liao J Y, Ma L M, Guo Y H, Zhang Y C, Zhou H, et al. Deep sequencing of human nuclear and cytoplasmic small RNAs reveals an unexpectedly complex subcellular distribution of miRNAs and tRNA 3′ trailers. PLoS One 5: e10563.
20. Ohrt T, Mutze J, Staroske W, Weinmann L, Hock J, et al. (2008) Fluorescence correlation spectroscopy and fluorescence cross-correlation spectroscopy reveal the cytoplasmic origination of loaded nuclear RISC in vivo in human cells. Nucleic Acids Res 36: 6439-6449.
21. Gibbings D J, Ciaudo C, Erhardt M, Voinnet O (2009) Multivesicular bodies associate with components of miRNA effector complexes and modulate miRNA activity. Nat Cell Biol 11: 1143-1149.
22. Lee Y S, Pressman S, Andress A P, Kim K, White J L, et al. (2009) Silencing by small RNAs is linked to endosomal trafficking. Nat Cell Biol 11: 1150-1156.
23. Kren B T, Wong P Y, Sarver A, Zhang X, Zeng Y, et al. (2009) MicroRNAs identified in highly purified liver-derived mitochondria may play a role in apoptosis. RNA Biol 6: 65-72.
24. Maniataki E, Mourelatos Z (2005) Human mitochondrial tRNAMet is exported to the cytoplasm and associates with the Argonaute 2 protein. Rna 11: 849-852.
25. Hornig-Do H T, Gunther G, Bust M, Lehnartz P, Bosio A, et al. (2009) Isolation of functional pure mitochondria by superparamagnetic microbeads. Anal Biochem 389: 1-5.
26. Cikaluk D E, Tahbaz N, Hendricks L C, DiMattia G E, Hansen D, et al. (1999) GERp95, a membrane-associated protein that belongs to a family of proteins involved in stem cell differentiation. Mol Biol Cell 10: 3357-3372.
27. Jakymiw A, Lian S, Eystathioy T, Li S, Satoh M, et al. (2005) Disruption of GW bodies impairs mammalian RNA interference. Nat Cell Biol 7: 1267-1274.
28. van Steensel B, van Binnendijk E P, Hornsby C D, van der Voort H T, Krozowski Z S, et al. (1996) Partial colocalization of glucocorticoid and mineralocorticoid receptors in discrete compartments in nuclei of rat hippocampus neurons. J Cell Sci 109 (Pt 4): 787-792.
29. Emanuelsson O, Brunak S, von Heijne G, Nielsen H (2007) Locating proteins in the cell using TargetP, SignalP and related tools. Nat Protoc 2: 953-971.
30. Claros M G, Vincens P (1996) Computational method to predict mitochondrially imported proteins and their targeting sequences. Eur J Biochem 241: 779-786.
31. Small I, Peeters N, Legeai F, Lurin C (2004) Predotar: A tool for rapidly screening proteomes for N-terminal targeting sequences. Proteomics 4: 1581-1590.
32. Bhasin M, Raghava G P (2004) ESLpred: SVM-based method for subcellular localization of eukaryotic proteins using dipeptide composition and PSI-BLAST. Nucleic Acids Res 32: W414-419.
33. Beitzinger M, Peters L, Zhu J Y, Kremmer E, Meister G (2007) Identification of human microRNA targets from isolated argonaute protein complexes. RNA Biol 4: 76-84.
34. Fleige S, Pfaffl M W (2006) RNA integrity and the effect on the real-time qRT-PCR performance. Mol Aspects Med 27: 126-139.
35. Hausser J, Benninger P, Rodak C, Jantscher Y, Wirth S, et al. (2009) MirZ: an integrated microRNA expression atlas and target prediction resource. Nucleic Acids Res 37: W266-272.
36. Ritchie W, Flamant S, Rasko J E mimiRNA: a microRNA expression profiler and classification resource designed to identify functional correlations between microRNAs and their targets. Bioinformatics 26: 223-227.
37. Kawaji H, Nakamura M, Takahashi Y, Sandelin A, Katayama S, et al. (2008) Hidden layers of human small RNAs. BMC Genomics 9: 157.
38. Pederson T Regulatory RNAs derived from transfer RNA? Rna 16: 1865-1869.
39. Lu M, Zhang Q, Deng M, Miao J, Guo Y, et al. (2008) An analysis of human microRNA and disease associations. PLoS One 3: e3420.
40. Pagliarini D J, Calvo S E, Chang B, Sheth S A, Vafai S B, et al. (2008) A mitochondrial protein compendium elucidates complex I disease biology. Cell 134: 112-123.
41. Ekins S, Nikolsky Y, Bugrim A, Kirillov E, Nikolskaya T (2007) Pathway mapping tools for analysis of high content data. Methods Mol Biol 356: 319-350.
42. Falkenberg M, Larsson N G, Gustafsson C M (2007) DNA replication and transcription in mammalian mitochondria. Annu Rev Biochem 76: 679-699.
43. Baskerville S, Bartel D P (2005) Microarray profiling of microRNAs reveals frequent coexpression with neighboring miRNAs and host genes. Rna 11: 241-247.
44. Politz J C, Hogan E M, Pederson T (2009) MicroRNAs with a nucleolar location. Rna 15: 1705-1715.
45. Yang J H, Li J H, Shao P, Zhou H, Chen Y Q, et al. starBase: a database for exploring microRNA-mRNA interaction maps from Argonaute CLIP-Seq and Degradome-Seq data. Nucleic Acids Res 39: D202-209.
46. Willkomm D K, Hartmann R K (2006) Intricacies and surprises of nuclear-mitochondrial co-evolution. Biochem J 399: e7-9.
47. Lynch M, Koskella B, Schaack S (2006) Mutation pressure and the evolution of organelle genomic architecture. Science 311: 1727-1730.
48. Sen G L, Blau H M (2005) Argonaute 2/RISC resides in sites of mammalian mRNA decay known as cytoplasmic bodies. Nat Cell Biol 7: 633-636.
49. Hock J, Meister G (2008) The Argonaute protein family. Genome Biol 9: 210.
50. Chen C Y, Zheng D, Xia Z, Shyu A B (2009) Ago-TNRC6 triggers microRNA-mediated decay by promoting two deadenylation steps. Nat Struct Mol Biol 16: 1160-1166.
51. Janowski B A, Huffman K E, Schwartz J C, Ram R, Nordsell R, et al. (2006) Involvement of AGO1 and AGO2 in mammalian transcriptional silencing. Nat Struct Mol Biol 13: 787-792.
52. Anderson S, Bankier A T, Barrell B G, de Bruijn M H, Coulson A R, et al. (1981) Sequence and organization of the human mitochondrial genome. Nature 290: 457-465.
53. Hock J, Weinmann L, Ender C, Rudel S, Kremmer E, et al. (2007) Proteomic and functional analysis of Argonaute-containing mRNA-protein complexes in human cells. EMBO Rep 8: 1052-1060.
54. Pare J M, Tahbaz N, Lopez-Orozco J, LaPointe P, Lasko P, et al. (2009) Hsp90 regulates the function of argonaute 2 and its recruitment to stress granules and P-bodies. Mol Biol Cell 20: 3273-3284.
55. Griffiths-Jones S, Grocock R J, van Dongen S, Bateman A, Enright A J (2006) miRBase: microRNA sequences, targets and gene nomenclature. Nucleic Acids Res 34: D140-144.
56. Kent W J (2002) BLAT—the BLAST-like alignment tool. Genome Res 12: 656-664.
57. Gasteiger E. HC, Gattiker A., Duvaud S., Wilkins M. R., Appel R. D., Bairoch A. (2005) Protein Identification and Analysis Tools on the ExPASy Server;
. (In) John M Walker (ed): The Proteomics Protocols Handbook, Humana Press: 571-607.
58. Gaines G, Attardi G (1984) Highly efficient RNA-synthesizing system that uses isolated human mitochondria: new initiation events and in vivo-like processing patterns. Mol Cell Biol 4: 1605-1617.
59. Asmann Y W, Stump C S, Short K R, Coenen-Schimke J M, Guo Z, et al. (2006) Skeletal muscle mitochondrial functions, mitochondrial DNA copy numbers, and gene transcript profiles in type 2 diabetic and nondiabetic subjects at equal levels of low or high insulin and euglycemia. Diabetes 55: 3309-3319.
60. Smith (1967) Preparation, properties and conditions for assay of mitochondria: Slaughter-house material, small scale. Methods in Enzymology 10: 81-86.
61. Olichon A, Emorine L J, Descoins E, Pelloquin L, Brichese L, et al. (2002) The human dynamin-related protein OPAL is anchored to the mitochondrial inner membrane facing the inter-membrane space. FEBS Lett 523: 171-176.
62. Antonsson B, Montessuit S, Sanchez B, Martinou J C (2001) Bax is present as a high molecular weight oligomer/complex in the mitochondrial membrane of apoptotic cells. J Biol Chem 276: 11615-11623.
63. Malka F, Aure K, Goffart S, Spelbrink J N, Rojo M (2007) The mitochondria of cultured mammalian cells: I. Analysis by immunofluorescence microscopy, histochemistry, subcellular fractionation, and cell fusion. Methods Mol Biol 372: 3-16.
64. Rozen S, Skaletsky H (2000) Primer3 on the WWW for general users and for biologist programmers. Methods Mol Biol 132: 365-386.
65. Sarkar M, Das S, Bandyopadhaya A, Ray K, Chaudhuri K (2005) Upregulation of human mitochondrial NADH dehydrogenase subunit 5 in intestinal epithelial cells is modulated by Vibrio cholerae pathogenesis. FEBS Lett 579: 3449-3460.
66. Bolte S, Cordelieres F P (2006) A guided tour into subcellular colocalization analysis in light microscopy. J Microsc 224: 213-232.
67. Adler J, Parmryd I (2007) Recent review on colocalization seem to misunderstand the Pearson correlation coefficient. J Microsc 227: 83; author reply 84-85.
68. Zuker M (2003) Mfold web server for nucleic acid folding and hybridization prediction. Nucleic Acids Res 31: 3406-3415.
69. Gruber A R, Bernhart S H, Hofacker I L, Washietl S (2008) Strategies for measuring evolutionary conservation of RNA secondary structures. BMC Bioinformatics 9: 122.
70. Hofacker I L (2003) Vienna RNA secondary structure server. Nucleic Acids Res 31: 3429-3431.
71. Zhang B H, Pan X P, Cox S B, Cobb G P, Anderson T A (2006) Evidence that miRNAs are different from other RNAs. Cell Mol Life Sci 63: 246-254.
72. Wang X (2008) miRDB: a microRNA target prediction and functional annotation database with a wild interface. Rna 14: 1012-1017.
73. Huang H Y, Chien C H, Jen K H, Huang H D (2006) RegRNA: an integrated web server for identifying regulatory RNA motifs and elements. Nucleic Acids Res 34: W429-434.
74. Miranda K C, Huynh T, Tay Y, Ang Y S, Tam W L, et al. (2006) A pattern-based method for the identification of MicroRNA binding sites and their corresponding heteroduplexes. Cell 126: 1203-1217.
75. Tan M G, Ooi L L, Aw S E, Hui K M (2004) Cloning and identification of hepatocellular carcinoma down-regulated mitochondrial carrier protein, a novel liver-specific uncoupling protein. J Biol Chem 279: 45235-45244.
76. Torchetti E M, Brizio C, Colella M, Galluccio M, Giancaspero T A, et al. Mitochondrial localization of human FAD synthetase isoform 1. Mitochondrion 10: 263-273.

1. A method for adjusting the expression of the mitochondrial genome in a human cell comprising a step of modulating the expression of a least one miRNA selected from the group consisting of hsa-miR-1973, hsa-miR-1275, hsa-miR-494, hsa-miR-513a-5p, hsa-miR-1246, hsa-miR-328, hsa-miR-1908, hsa-miR-1972, hsa-miR-1974, hsa-miR-1977, hsa-miR-638, hsa-miR-1978 and hsa-miR-1201.
